(19) United States
(12) Patent Application Publication
(10) Pub. No.: US 2020/0230393 A1
(43) Pub. Date: Jul. 23, 2020

(54) HARVEY E/R (EXPANDABLE/RETRACTABLE) NASAL OXYGEN CANNULA SYSTEM AND METHOD OF USE

(71) Applicant: Meldrum J Harvey, Mineola, TX (US)

(72) Inventor: Meldrum J Harvey, Mineola, TX (US)

(21) Appl. No.: 16/265,069

(22) Filed: Feb. 1, 2019

Related U.S. Application Data
(60) Provisional application No. 62/794,329, filed on Jan. 18, 2019.

(51) Int. Cl. A61M 39/08; A61M 16/06; A61M 16/08

(52) U.S. Cl. CPC A61M 39/08; A61M 16/0875; A61M 16/0672

(57) ABSTRACT

A Harvey E/R (expandable/retractable) nasal oxygen cannula system enables a person that needs assistance with additional air or oxygen to receive this assistance without creating other hazards or limits on their movement. The system uses a retractable tube that stretches or other wise expands and contracts as the person increase or decreases their distance from the source of the assisted air or oxygen.

### BACKGROUND

### 1. Field of the Invention

The present invention relates generally to assisted respiratory systems, and more specifically, to an expandable/retractable nasal oxygen cannula system for delivering oxygen gas directly to the openings of the nose while not impeding the mobility of the person intaking the oxygen.

### 2. Description of Related Art

Assisted respiratory systems are well known in the art and are effective means to increase the oxygen intake of a person. For example, FIG. 1 depicts a conventional supplemental oxygen delivery system 101 having an oxygen supply 103 with a nasal cannula 105 attached by a tube 107. During use, a person 109 places the nasal cannula 105 near or in their nose 111 and then runs the oxygen supply to allow oxygen to arrive in the nose 111 through the tube 107.

One of the problems commonly associated with system 101 is its limited use. For example, the mobility of the person 109 is limited by their ability to move the oxygen supply 103 which must be carried, pulled or otherwise moved along with the person 109. The oxygen supply 103 is commonly placed on a cart, or provided by a portable concentrator device, to allow the person 109 to move the oxygen supply. It is common for those who use the supplemental oxygen delivery system 101 to have other physical limitations and that the burden of moving the system 101 complicates the mobility of the person 109.

In addition, the tube 107 creates limitations on mobility by creating a trip hazard or its limited length. When the tube 107 is long it will fall to or near the ground where it catches the feet or lower legs of the person 109. Conventionally, the length of the tube 107 is shortened to reduce this risk further limiting the distance a person 109 may be from the oxygen supply 103.

Accordingly, although great strides have been made in the area of supplemental oxygen delivery system, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a front view of a common supplemental oxygen delivery system;

FIG. 2 is a front view of a Harvey E/R (expandable/retractable) nasal oxygen cannula system in accordance with a preferred embodiment of the present application;

FIGS. 3A and 3B are top views of the system of FIG. 2 in use,

FIG. 4 is a front view of an alternative embodiment of the system of FIG. 2;

FIG. 5 is a cross-sectional side view of the rotational joint of FIG. 4;

FIG. 6 is a cross sectional side view of an alternative embodiment of the retractable tube of FIG. 2; and

FIG. 7 is a flowchart of the preferred method of use of the system of FIG. 2.

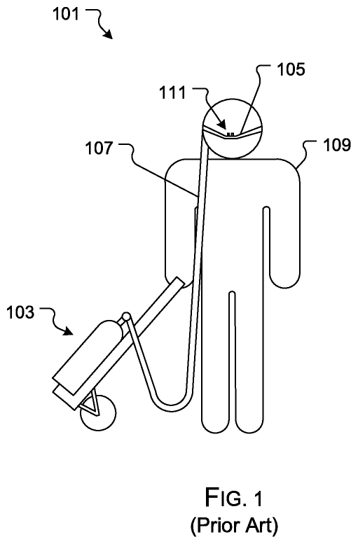

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional supplemental oxygen delivery systems. Specifically, the invention of the present application increases the mobility of a person needing supplemental air or oxygen. In addition, the invention reduces the trip hazard presented by the tube. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

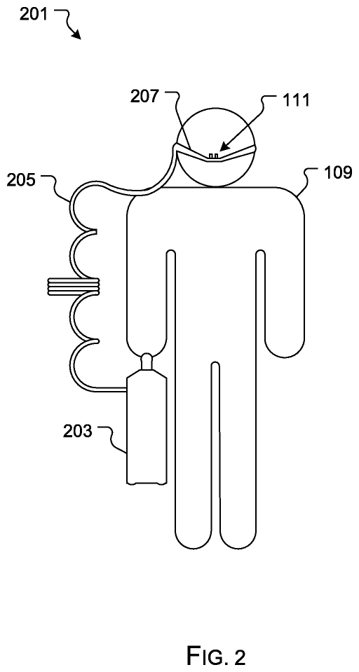

Referring now to the drawings wherein like reference characters identify corresponding or similar elements throughout the several views, FIG. 2 depicts a top view of a Harvey E/R (expandable/retractable) nasal oxygen cannula system in accordance with a preferred embodiment of the present application. It will be appreciated that system 201 overcomes one or more of the above-listed problems commonly associated with conventional supplemental oxygen delivery systems.

In the contemplated embodiment, system 201 includes a portable fluid supply device 203 configured to produce, mix, filter or otherwise deliver oxygen or a mixture thereof through a retractable tube 205 to the nasal opening 111 of a person 109 via a cannula 207. While the preferred embodiment includes a portable fluid supply device 203 it is contemplated that a stationary fluid supply could be used with the system. It is also contemplated that any fluid could be used with the system and that it should not be limited in scope to merely oxygen or air.

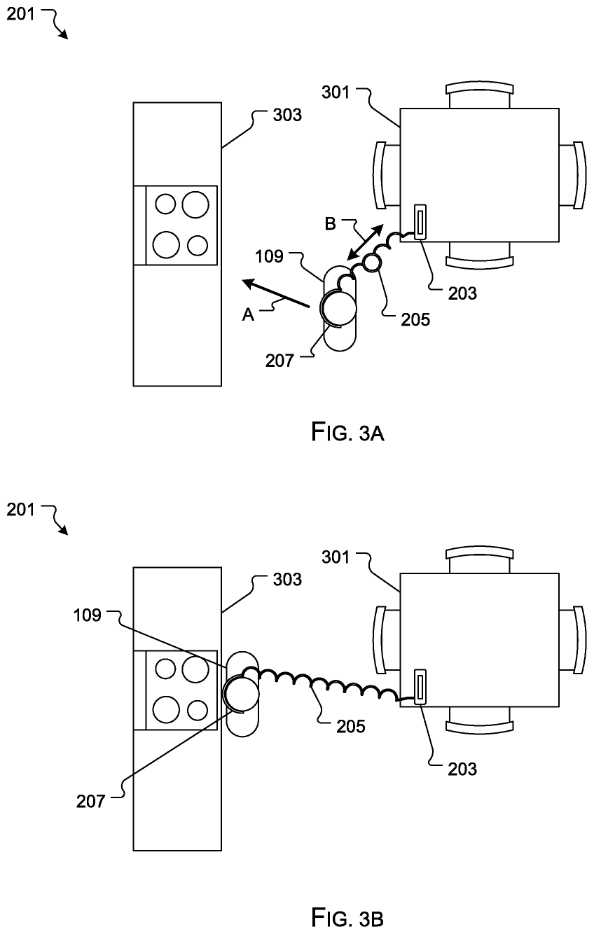

In use, the portable fluid supply device 203 is carried with the person 109 or placed centrally, such as on a table 301, in a room as depicted in FIGS. 3A and 3B. As the person 109 moves with respect to the portable fluid supply device 203, such as to a counter 303 or other space, as shown by motion A the retractable tube 205 expands or retracts as depicted by motion B.

It should be appreciated that one of the unique features believed characteristic of the present application is that the retractable tube 205 does not become a trip hazard for the person 109 allowing for greater mobility.

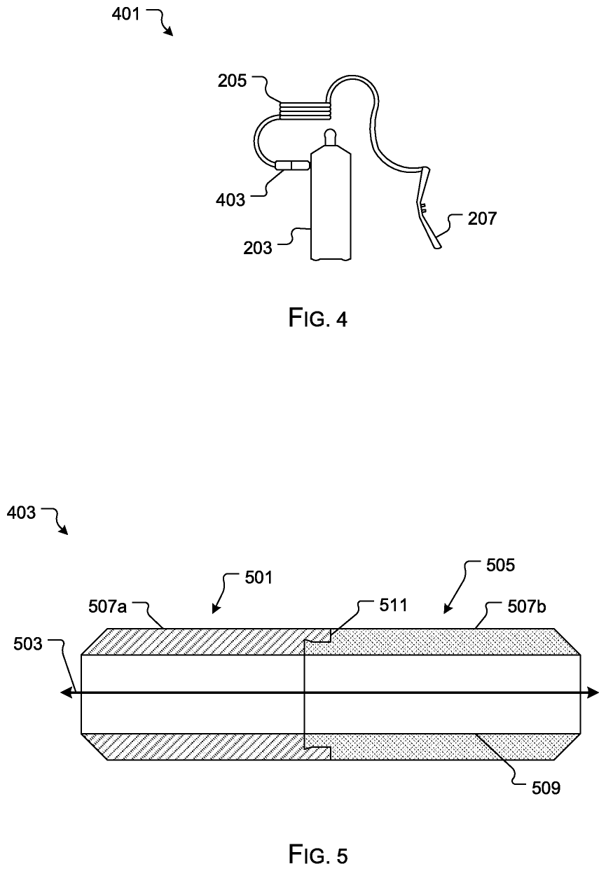

In an alternative embodiment it is contemplated that the retractable tube 205 could be attached and in fluid communication with the portable fluid supply device 203 via rotational joint 403 as depicted in FIG. 4. It will be appreciated that the rotational joint 403 reduces the twist in the retractable tube 205 further reducing the burden on the person 109 using system 201.

The rotational joint 403 includes a first half 501 that rotates along axis 503 with respect to a second half 505 via joint 511. The first half 501 and second half 505 include a body 507 with a passageway 509 through which a fluid may flow. The first half 501 attaches to the second half 505 via joint 511.

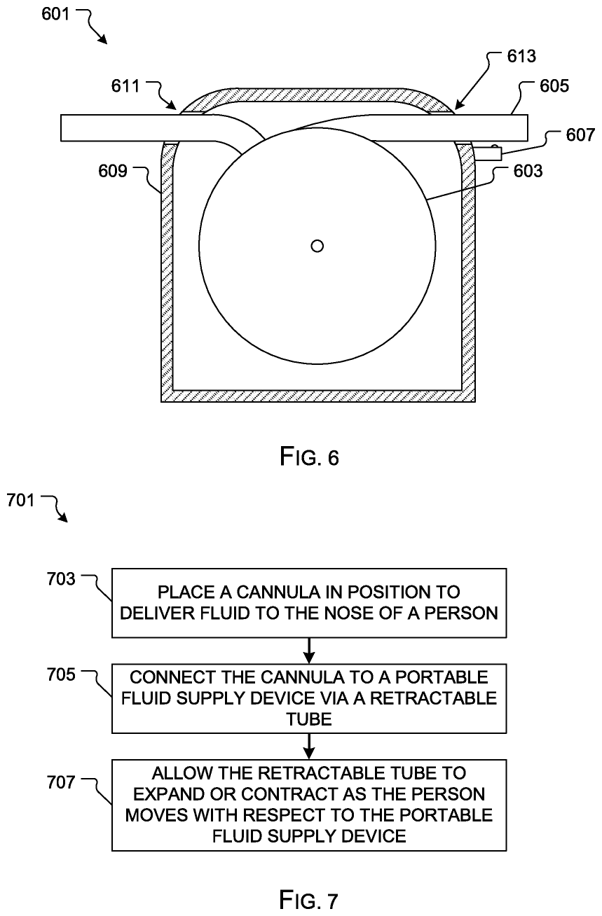

It is contemplated that any method of enabling the retractable tube 205 could be used and that while the present embodiment depicts coils other alternatives exist, for example referring now to FIG. 6 a motorized spindle 603 is contemplated. Spindle 603 is configured to wind and unwind tubing 605 as motion is detected by sensor 607. The motorized spindle 603 and tube 605 are contemplated to be stored in a housing 609. The tube 605 entering the housing 609 through a first opening 611 therein and exiting therefrom via a second opening 613 therein.

Referring now to FIG. 7 the preferred method of use of the system 201 is depicted. Method 701 includes placing a cannula in position to deliver fluid to the nose of a person 703, connecting the cannula to a portable fluid supply device via a retractable tube 705, allowing the retractable tube to expand or contract as the person moves with respect to the portable fluid supply device 707.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed:

1. A Harvey E/R (expandable/retractable) nasal oxygen cannula system comprising:
a cannula in fluid communication with a fluid supply device via at least one retractable tube;
wherein the retractable tube is configured to minimize its overall length; and
wherein the cannula is enabled to move with respect to the fluid supply device without creating trip hazards.

2. The system of claim 1 wherein the retractable tube is attached to the cannula via at least one rotational joint.

3. The system of claim 1 wherein the retractable tube comprises a coiled tube.

4. The system of claim 1 wherein the retractable tube is enabled by mechanical means.

5. The system of claim wherein oxygen is passed through the retractable tube.

5. The method of preventing trip, fall or snag hazards to a person using a cannula given the system of claim 1, comprising:
placing a cannula in position to deliver fluid to the nose of a person;
connecting the cannula to a portable fluid supply device via a retractable tube;
allowing the retractable tube to expand or contract as the person moves with respect to the portable fluid supply device.
